(19) United States
(12) Patent Application Publication
(10) Pub. No.: US 2026/0090987 A1
(43) Pub. Date: Apr. 2, 2026

(54) POUCH DISPENSING OF LOCAL ANESTHETIC

(71) Applicant: Andrew ALBEE, Suncook, NH (US)

(72) Inventor: Andrew ALBEE, Suncook, NH (US)

(21) Appl. No.: 19/347,190

(22) Filed: Oct. 1, 2025

Related U.S. Application Data
(60) Provisional application No. 63/702,424, filed on Oct. 2, 2024.

(51) Int. Cl. A61K 9/00; A61K 31/245; A61K 47/38

(52) U.S. Cl. CPC A61K 9/009; A61K 9/006; A61K 31/245; A61K 47/38

(57) ABSTRACT

An apparatus for dispensing topical anesthetic is described. The apparatus includes a pouch, a cover material, and a topical anesthetic. The pouch includes a containing material, and the pouch contains the topical anesthetic. A method of administering topical oral anesthetic using the pouch is also described.

### RELATED APPLICATIONS

This application claims the benefit under 35 U.S.C. § 119 (e) of U.S. Provisional Patent Application No. 63/702,424, filed on 2 Oct. 2024, the contents of which are incorporated herein by reference in its entirety.

### TECHNICAL FIELD

This disclosure relates generally to delivery of topical anesthetics and, more specifically, to an apparatus and method for delivery of topical anesthetics to the oral cavity.

### BACKGROUND

Many products are currently available for topical, oral pain relief. Some of these products come in a cream or gel form, which can be inconvenient for users to apply as the product moves around uncontained.

### SUMMARY

In one aspect, an apparatus is provided. The apparatus may be a pouch including a topical anesthetic and a cover material. The cover material may be insoluble in water and may be permeable to water. The cover material may include a polysaccharide. The cover material may be made of cellulose, such as paper or cotton. The cover material may also be a synthetic, such as polyolefin or PTFE.

The topical anesthetic may include an amino ester anesthetic. The topical anesthetic may be benzocaine. The benzocaine may be in a powdered form. The apparatus may further include a filler contained by the pouch.

The pouch may be shaped for buccal administration. The pouch may be shaped for sublingual administration. The pouch may be shaped as a cylinder that is closed on both ends.

In another aspect, a method is provided. The method includes inserting a pouch containing a topical anesthetic into an oral cavity, aligning the pouch with an affected area of the oral cavity, and retaining the pouch over the affected area. The method may include removing the pouch from the oral cavity. The method may include retaining the pouch over the affected area until all the topical anesthetic has been released from the pouch. The method may include retaining the pouch over the affected area until a user no longer feels pain or discomfort associated with the affected area. The method may include retaining the pouch over the affected area until the affected area is numbed enough for injection of a local anesthetic. The method may include injecting a local anesthetic in the affected area.

The systems, devices, and methods described herein may be used separately or together, and components or techniques described in relation to one system or method are capable of being implemented with the others. The subject matter of this application may involve, in some cases, interrelated products, alternative solutions to a particular problem, and/or a plurality of different uses of a single system or article.

### BRIEF DESCRIPTION OF THE DRAWINGS

The above-mentioned and other features of this disclosure, and the manner of attaining them, will become more apparent and better understood by reference to the following description of embodiments described herein taken in conjunction with the accompanying drawings.

FIG. 1 illustrates one embodiment of a pouch for administering topical anesthetic.

FIG. 2 illustrates an open pouch with a topical anesthetic and a filler, according to an embodiment.

FIG. 3A illustrates an affected area of an oral cavity.

FIG. 3B illustrates a pouch placed over an affected area of an oral cavity, according to an embodiment.

### DETAILED DESCRIPTION

In dentistry and related fields there is often a need to numb an area of the oral cavity topically before administration of an injectable anesthetic. Typically, this topical numbing is done by a product in a cream or gel form. Products in this form can be challenging for a user to apply as they are uncontained and may move around the oral cavity away from an affected area. The actual dosage applied can vary depending on the amount of contact of the gel or cream with affected area.

According to an embodiment, this disclosure describes a pouch that provides for easy administration of topical anesthetic to the oral cavity. The pouch contains a topical anesthetic to provide pain relief and/or numbing to an affected area of the oral cavity. The pouch includes a containing material to control the release of the topical anesthetic. The containing material may be insoluble in water so that the pouch does not dissolve when situated in the oral cavity. The pouch may also contain fillers that enhance a user's experience. Flavorings and sweeteners can provide a pleasant taste to the user. Antiseptic agents can help to prevent the growth of unwanted microorganisms in the oral cavity. Stabilizers or preservatives can help to keep the product fresh for use. pH adjusting agents can be used to maintain a healthy pH of the oral cavity during use of the product. The pouch may be shaped as a cylinder closed on both ends to form a flat packet that easily fits into a user's oral cavity. The pouch may be shaped for buccal administration or sublingual administration to treat an affected area in different parts of the oral cavity. For instance, the pouch can be substantially cylindrical, and may have a length to width ratio of greater than 1:1, greater than 2:1 or greater than 3:1. In cross-section along a longitudinal axis, the pouch can have a height that is greater than its width, the ratio of which can be, for example, greater than 1.5:1 or greater than 2:1. The pouch may be malleable, meaning that it can be easily shaped to fit comfortably in the patient's mouth. This malleability allows a large portion of the surface of the pouch to be in contact with the gingiva, for example. In some cases, the pouch may be formed into a shape that provides for more than 50% of the surface area of the pouch to be in contact with a membrane of the oral cavity. In other cases, the amount of contact can be greater than 75%, greater than 80% or greater than 90%.

In some embodiments the pouch can be made of a single material that is consistent over the entire surface of the pouch. In other embodiments a first portion of the pouch can be permeable to the topical anesthetic and a second portion can be impermeable. This allows the practitioner to target a specific area of the oral cavity without administering more topical anesthetic than is necessary for the numbing required. In some cases, one longitudinal side of the pouch can be permeable and the opposed longitudinal side can be impermeable to the topical anesthetic.

In another aspect, a method is provided for administering topical anesthetic to the oral cavity. To use a pouch, the practitioner inserts the pouch into the patient's oral cavity and aligns the pouch with an area to be de-sensitized. The area can be, for example, a location where the practitioner wishes to administer an injection. An affected area can include a damaged tooth or root, a lesion, a sore, an ulcer, an injury caused by orthodontic equipment, or an area to be treated further with injectable anesthetic. The pouch can be retained over the affected area until a user notices the numbness or no longer feels pain or discomfort associated with the affected area. Alternatively, the pouch can be retained over the affected area until all, or substantially all, of the topical anesthetic has been released from the pouch. The pouch can also be retained over the affected area until the area is numb enough to administer injectable anesthetic. An injectable anesthetic can subsequently be administered to the affected area. The pouch can be removed from the oral cavity after administration of the topical anesthetic is complete.

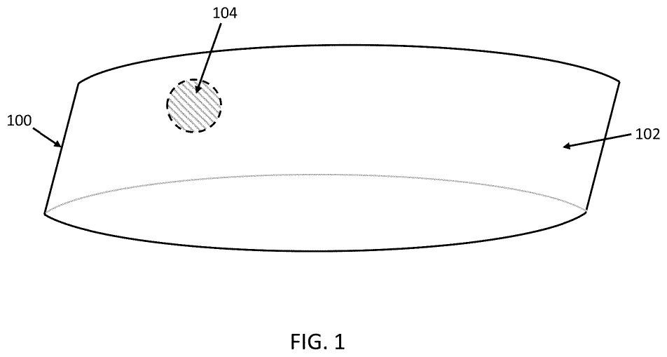

FIG. 1 provides a schematic figure of one embodiment of a pouch 100 having a cover material 102 enclosing a topical anesthetic 104. Pouch 100 may also contain a material that is adhered to topical anesthetic 104 and can provide enough bulk so that the anesthetic can be easily measured out. Cover material 102 may be insoluble in water so that pouch 100 does not dissolve in the oral cavity. Cover material 102 may be synthetic or natural. In some cases, cover material 102 can comprise cellulose or another polysaccharide. Topical anesthetic 104 is placed inside pouch 100 and the pouch may be sealed, such as by folding an adhesive or a complementary telescoping pouch. Topical anesthetic 104 can comprise any suitable amino ester anesthetic for administration to the oral cavity such as tetracaine or benzocaine. Topical anesthetic 104 can be in a liquid, gel, or powder form. Topical anesthetic 104 may also include any suitable amino amide anesthetic for administration to the oral cavity such as lidocaine, prilocaine, mepivacaine, bupivacaine, levobupivacaine, or ropivacaine.

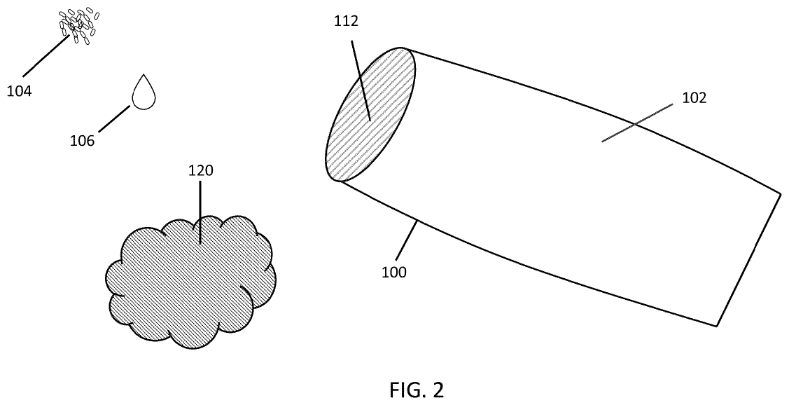

FIG. 2 provides a view of an empty pouch 100 showing an opening 112 at one end of cover material 102. Cover material 102 controls the release of topical anesthetic 104 and, in some embodiments, a filler 120, from an interior of pouch 100 to the oral cavity. Filler 120 can be an inert material whose purpose is to provide bulk to the pouch so that it can maintain its shape. The anesthetic can be, for example, powder 104a or liquid 104b. In accordance with some embodiments, filler 120 can be mixed with topical anesthetic 104. Filler 120 may be added to pouch 100 in various embodiments and can serve as a conveyance means for loading anesthetic 104 into pouch 100 or can occupy space in pouch 100 to provide the desired shape and size of the pouch. Additional additives such as flavorings and sweeteners can provide a pleasant taste to the user. Antiseptic agents can prevent the growth of unwanted microorganisms in the oral cavity. Stabilizers or preservatives can help to keep the product fresh and ready for use. pH adjusting agents can be used to maintain a healthy pH of the oral cavity during use of the product. Topical anesthetic 104 can be loaded into the pouch as a pure powder, mixed with a filler, as a liquid, or dissolved in a solvent, such as water or alcohol, to name a few examples.

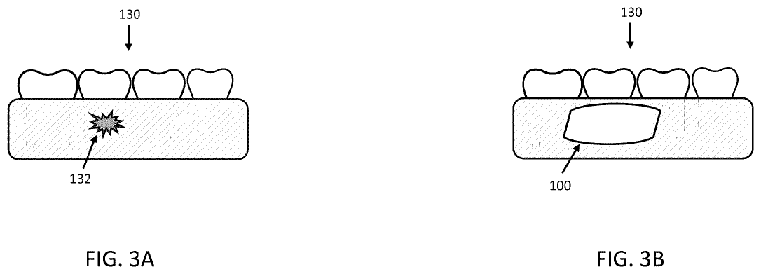

FIG. 3A provides a view of an affected area 132 inside an oral cavity 130. FIG. 3B illustrates placing pouch 100 over the affected area 132 to administer topical anesthetic 104. Affected area 132 may be an area of oral cavity 130 that requires numbing or pain relief treatment. Affected area 132 may include a lesion, sore, ulcer, injury caused by orthodontic equipment, or an area to be treated further with injectable anesthetic. Pouch 100 can be retained over affected area 132 until adequate numbness is achieved or, for example, until a user no longer feels pain or discomfort associated with the affected area. This embodiment of the method can be useful to users trying to relieve pain or discomfort caused by minor injuries from orthodontic equipment or other minor injuries to the oral cavity. Pouch 100 can also be retained until substantially all of topical anesthetic 104 has been released. This allows for more control over the amount of topical anesthetic 104 delivered as a known quantity of topical anesthetic 104 can be contained within pouch 100. The rate of migration of topical anesthetic 104 to the affected area can be controlled by, for example, the permeability of cover material 102, the level of attachment of topical anesthetic 104 to a filler material, or the amount of moisture that infiltrates pouch 100. If pouch 100 is being used in preparation for a local or injectable anesthetic, pouch 100 can be retained until the affected area is numbed enough for injection. A local or injectable anesthetic can subsequently be administered, and the used pouch can be disposed of.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, that is, elements that are conjunctively present in some cases and disjunctively present in other cases. Other elements may optionally be present other than the elements specifically identified by the “and/or” clause, whether related or unrelated to those elements specifically identified, unless clearly indicated to the contrary.

What is claimed is:

1. An apparatus for oral administration of an anesthetic comprising:
a topical anesthetic;
a cover material forming a pouch and containing the topical anesthetic, wherein at least a portion of the cover material is permeable to the topical anesthetic.

2. The apparatus of claim 1, wherein the pouch is shaped to be placed against the gingiva of a patient.

3. The apparatus of claim 1, wherein the cover material comprises a polysaccharide.

4. The apparatus of claim 1, wherein the cover material comprises cellulose.

5. The apparatus of claim 1, wherein the topical anesthetic comprises an amino ester anesthetic.

6. The apparatus of claim 1, wherein the topical anesthetic is benzocaine.

7. The apparatus of claim 1, wherein the topical anesthetic is in a powdered form.

8. The apparatus of claim 1, further comprising a filler.

9. The apparatus of claim 1, wherein the pouch is shaped for buccal administration.

10. The apparatus of claim 1, wherein the pouch is shaped for sublingual administration.

11. The apparatus of claim 1, wherein the pouch is malleable.

12. The apparatus of claim 1 wherein at least a portion of the cover material is impermeable to the topical anesthetic.

13. A method of administering topical, oral anesthetic comprising:
inserting a pouch containing a topical anesthetic into an oral cavity;
aligning the pouch with an affected area of the oral cavity;
allowing the topical anesthetic to diffuse through a cover material of the pouch; and
removing the pouch from the oral cavity after the topical anesthetic has diffused through the cover material.

14. The method of claim 13, wherein the topical anesthetic diffuses through only one portion of the cover material.

15. The method of claim 13, further comprising retaining the pouch over the affected area.

16. The method of claim 15, wherein retaining the pouch over the affected area comprises retaining the pouch over the affected area until all the topical anesthetic has been released from the pouch.

17. The method of claim 15, wherein retaining the pouch over the affected area comprises retaining the pouch over the affected area until a user cannot feel prodding to the area.

18. The method of claim 15, wherein retaining the pouch over the affected area comprises retaining the pouch over the affected area until the affected area is sufficiently numb for injection of a local anesthetic.

19. The method of claim 18, further comprising injecting the local anesthetic in the affected area.
